(19) United States
(12) Patent Application Publication
(10) Pub. No.: US 2021/0252202 A1
(43) Pub. Date: Aug. 19, 2021

(54) SMART BREAST PUMP WITH MASSAGE COURSE GENERATING FUNCTION

(71) Applicant: CIMILRE CO., LTD., Cheonan-si (KR)

(72) Inventor: Sang Ha KIM, Seoul (KR)

(73) Assignee: CIMILRE CO., LTD., Cheonan-si (KR)

(21) Appl. No.: 17/105,450

(22) Filed: Nov. 25, 2020

(30) Foreign Application Priority Data
Feb. 19, 2020 (KR) 10-2020-0020098

(51) Int. Cl. A61M 1/06

(52) U.S. Cl. CPC A61M 1/066; A61M 2205/50; A61M 2205/8206

(57) ABSTRACT

The present invention relates to a smart breast pump with a massage course generating function including: a close contact unit attached to the user's breast; an expression unit for repeatedly producing expression pressures patterned by the user's input to control an internal pressure of the close contact unit; and a connection unit for connecting the close contact unit to the expression unit to have a relation with the production of the expression pressures in the close contact unit and the discharge of breast milk expressed, wherein through an input part mounted on the expression unit, any one of an expression mode for expressing the breast milk and a massage mode for massaging the breast is selected.

### CROSS REFERENCE TO RELATED APPLICATION OF THE INVENTION

The present application claims the benefit of Korean Patent Application No. 10-2020-0020098 filed in the Korean Intellectual Property Office on Feb. 19, 2020, the entire contents of which are incorporated herein by reference.

### BACKGROUND OF THE INVENTION

### Field of the Invention

The present invention relates to a breast pump with a massage course generating function by means of a user's input, and more particularly, to a smart breast pump with a massage course generating function that is capable of repeatedly producing expression pressures with strengths and patterns required through a user's input to perform the user's breast massage and storing all input while being operated to reproduce the same massage course as before, and generating and reproducing a massage course corrected through the input generated while a massage course stored therein is being reproduced.

### Background of the Related Art

Generally, a mother who gives birth to a baby feeds breast milk to her baby, but if an amount of breast milk fed to her baby is not sufficient or the breast milk excessively remains in her breasts, the breasts become hard to thus cause breast engorgement with severe pain.

Accordingly, the mother has to feed a sufficient amount of breast milk to her baby 8 to 12 times per day, or the breast milk has to be discharged artificially from her breasts. To do this, breast pumps having various shapes have been used.

In the past, on the other hand, the breast pump is controlled manually by the mother or another person to express the breast milk, but recently, breast pumps that express breast milk automatically have been widely used. Furthermore, the breast pump having a massage function has been used and developed to provide gentle flow of breast milk and the breast pain relief of the mother.

Conventional breast pumps are disclosed in Korean Patent Application Laid-open No. 10-2004-0071258 entitled “breast pump system”, Korean Patent No. 10-0755897 entitled “programmable breast pump”, and Korean Patent Application Laid-open No. 10-2019-0011044 entitled “programmable breast pump”.

The conventional breast pump system as disclosed in Korean Patent Application Laid-open No. 10-2004-0071258 is capable of controlling strengths of a positive pressure and a negative pressure applied to a mother's breast by means of a controller connected to a pressure source and adjusting a pressure period, and the conventional programmable breast pump as disclosed in Korean Patent No. 10-0755897 is capable of inputting various programs to a controller by means of an interface, thereby providing a suction method for a painful nipple, a suction method for increasing breast milk, a suction method improved generally, and a suction method for stimulating nipples.

According to the conventional breast pumps as mentioned above, however, only the expression pressures of the breast pump are controlled in real time by the user, or only any one program is selected among the various programs provided, so that there are limitations in allowing a user to directly generate a massage course optimized to her and in also allowing the user to repeatedly reproduce the massage course generated.

To solve the above-mentioned problems, the conventional programmable breast pump as disclosed in Korean Patent Application Laid-open No. 10-2019-0011044 is capable of generating and storing programs directly by a user, reproducing the stored programs, and performing selected deletion, selected correction, and selected addition according to situations, thereby generating the programs optimized to the user. In this case, however, the programs have to be previously set by the user.

Furthermore, the conventional programmable breast pump can change the pressure or cycle generated through the reproduction of the program in real time, but there is a limitation in reproducing a program in which the changed condition is applied again. So as to apply the changed condition to the program, accordingly, reproducing the program has to stop or selected correction has to be tried after the reproducing is finished. In this case, however, the changed program may not be completely the same as the program reproduced before.

Accordingly, there is a definite need for a breast pump capable of allowing a user to directly generate and repeatedly reproduce a massage course optimized to her, allowing the whole or portion of the massage course reproduced to be corrected simply, and allowing the corrected course to be reproduced again.

### SUMMARY OF THE INVENTION

Accordingly, the present invention has been made in view of the above-mentioned problems occurring in the related art, and it is an object of the present invention to provide a smart breast pump with a massage course generating function that is capable of repeatedly producing expression pressures with strengths and patterns required through a user's input to perform the user's breast massage and storing all input while being operated to reproduce the same massage course as before, and generating and reproducing a massage course corrected through the input generated while a massage course stored therein is being reproduced, thereby overcoming the limitations in allowing the user to directly generate a massage course optimized to her and in also allowing the user to repeatedly reproduce the massage course generated, which have occurred in the conventional breast pumps wherein only the expression pressures of the breast pump are controlled in real time by the user or only any one program is selected among the various programs provided, and also thereby overcoming the limitations in reproducing a program in which the changed condition is applied again and in that the changed program may not be completely the same as the program reproduced before, which have occurred in another conventional breast pump wherein the programs have to be previously set by the user and it is possible only to temporarily change the program during the reproduction of the program.

To accomplish the above-mentioned object, according to the present invention, there is provided a breast pump with a massage course generating function, including: a close contact unit attached to the user's breast; an expression unit for repeatedly producing expression pressures patterned by the user's input to control an internal pressure of the close contact unit; and a connection unit for connecting the close contact unit to the expression unit to have a relation with the production of the expression pressures in the close contact unit and the discharge of breast milk expressed, wherein through an input part mounted on the expression unit, any one of an expression mode for expressing the breast milk and a massage mode for massaging the breast is selected, so that if the massage mode is selected in a state where a massage course is not generated, various kinds of input signals generated through the input part are stored in order as time passes until a given set time elapses thus to generate and store a new massage course; if the massage mode is selected in a state where a massage course is stored, the stored massage course is reproduced; and from the time point where a new input signal is generated while the stored massage course is being reproduced, other input signals inclusive of the new input signal are stored in order as time passes to thus generate another new massage course.

According to the present invention, desirably, if an input signal for stopping driving the power supply part is generated while a new massage course is being generated through the input part, a massage course inclusive of one or more input signals previously inputted is immediately generated and stored; if an input signal for stopping driving the smart breast pump is generated to stop producing the expression pressure while the massage course stored is being reproduced, the massage course stored is corrected to a massage course wherein patterns inputted after the production of the expression pressure has stopped are deleted; and after the massage course has been corrected, if an input signal for driving the smart breast pump is first generated, the expression pressure produced at the time point when the production of the expression pressure has stopped is produced exactly until the massage course is finished.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIGS. 1A to 1D are perspective views showing sequential coupling processes of a smart breast pump with a massage course generating function according to the present invention;

FIG. 2 is a perspective view showing a close contact unit of the smart breast pump with a massage course generating function according to the present invention;

FIG. 3 is a sectional view showing a flow of breast milk expressed through the smart breast pump with a massage course generating function according to the present invention;

FIGS. 4A and 4B are perspective views showing a connection unit of the smart breast pump with a massage course generating function according to the present invention;

FIGS. 5A and 5B are plan and bottom views showing an expression unit of the smart breast pump with a massage course generating function according to the present invention; and

FIG. 6 is a perspective view showing an example of an expression hose used for the smart breast pump with a massage course generating function according to the present invention.

### DETAILED DESCRIPTION OF THE EMBODIMENTS

The present invention relates to a breast pump with a massage course generating function by means of a user's input, the breast pump including: a close contact unit attached to the user's breast; an expression unit for repeatedly producing expression pressures patterned by the user's input to control an internal pressure of the close contact unit; and a connection unit for connecting the close contact unit to the expression unit to have a relation with the production of the expression pressures in the close contact unit and the discharge of breast milk expressed, wherein through an input part mounted on the expression unit, any one of an expression mode for expressing the breast milk and a massage mode for massaging the breast is selected, so that if the massage mode is selected in a state where a massage course is not generated, various kinds of input signals generated through the input part are stored in order as time passes until a given set time elapses thus to generate and store a new massage course; if the massage mode is selected in a state where a massage course is stored, the stored massage course is reproduced; and from the time point where a new input signal is generated while the stored massage course is being reproduced, other input signals inclusive of the new input signal are stored in order as time passes to thus generate another new massage course.

Hereinafter, the present invention will be in detail explained with reference to the attached drawings.

First, the close contact unit 100 is adapted to come into close contact with a breast of a user as a mother to allow the breast milk introduced thereinto through expression to be moved and discharged, and accordingly, the close contact unit 100 is necessarily provided for all of breast pumps for expressing breast milk.

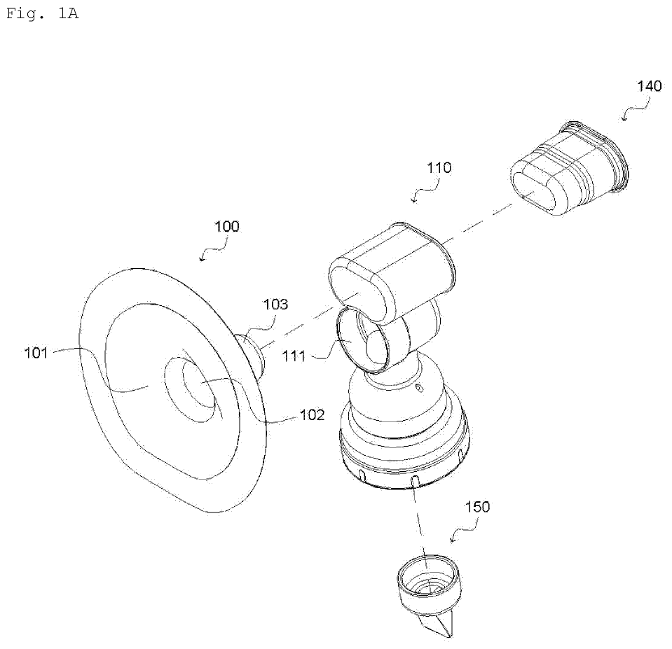

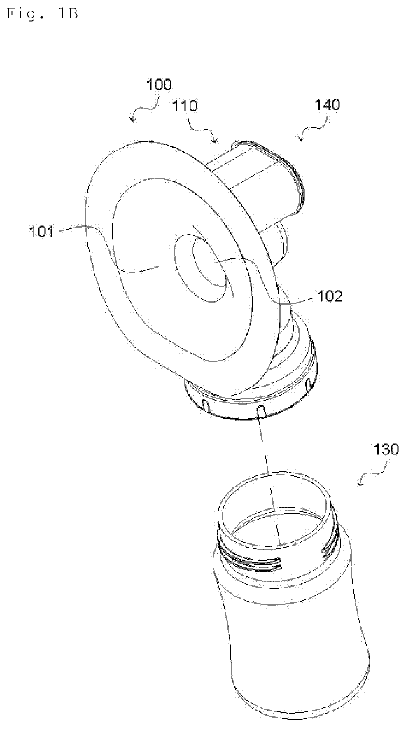

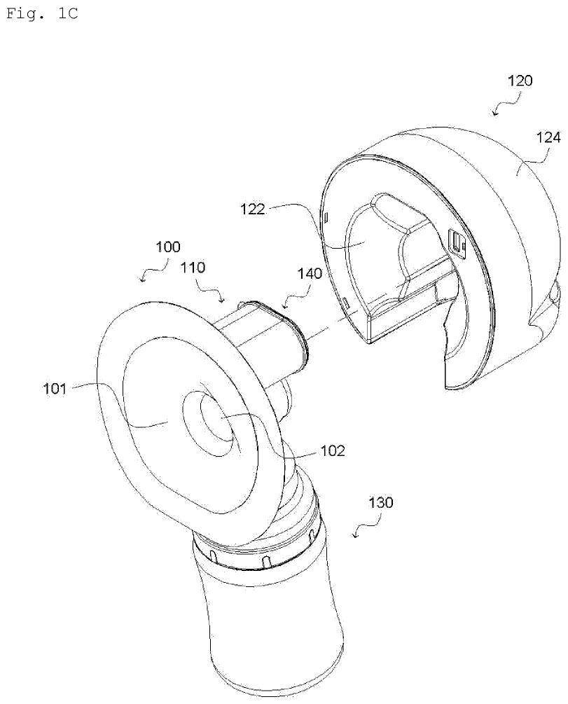

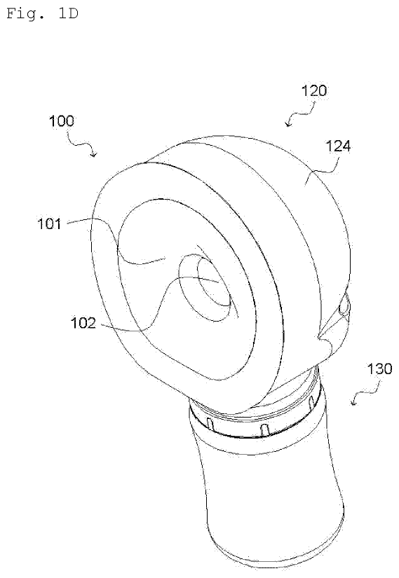

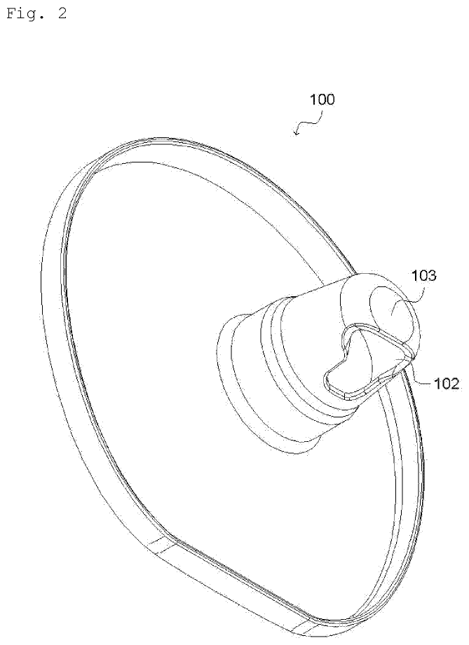

In detail, the close contact unit 100 has various shapes capable of coming into close contact with the user's breast to express and discharge breast milk, but desirably, the close contact unit 100 generally has the shape of a cone with an insertion groove 101 formed on one side thereof to insert the user's breast thereinto, as shown in FIGS. 1A to 1D, and a fastening portion 103 protruding from the other side thereof to allow an end portion of an internal passage 102 connected to the insertion groove 101 to be open downward therefrom, as shown in FIG. 2.

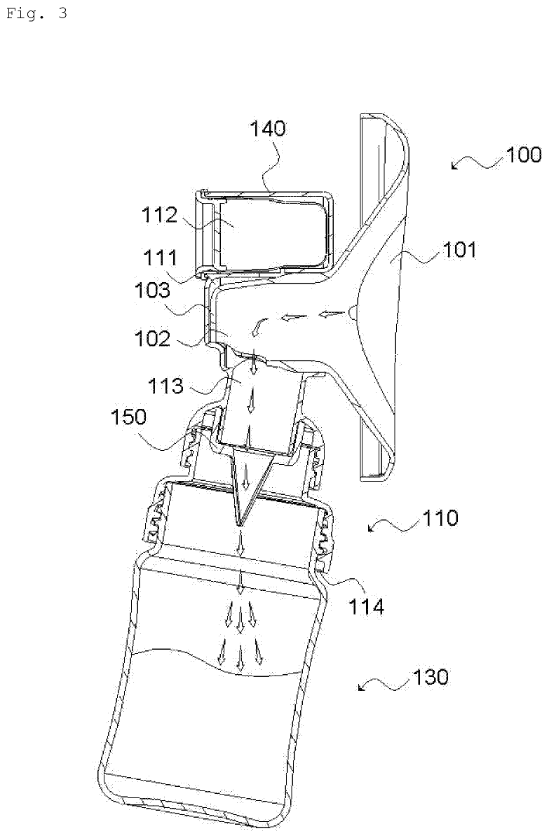

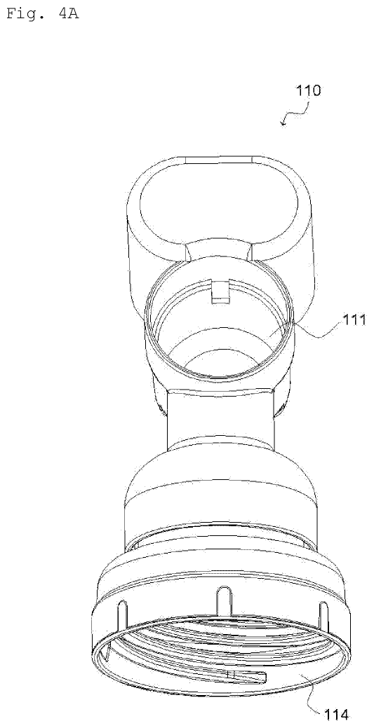

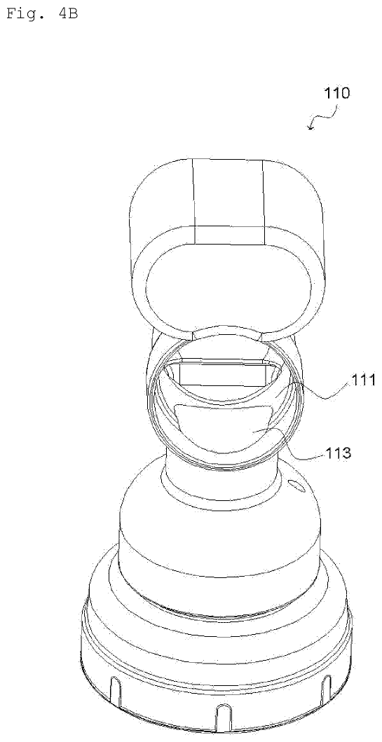

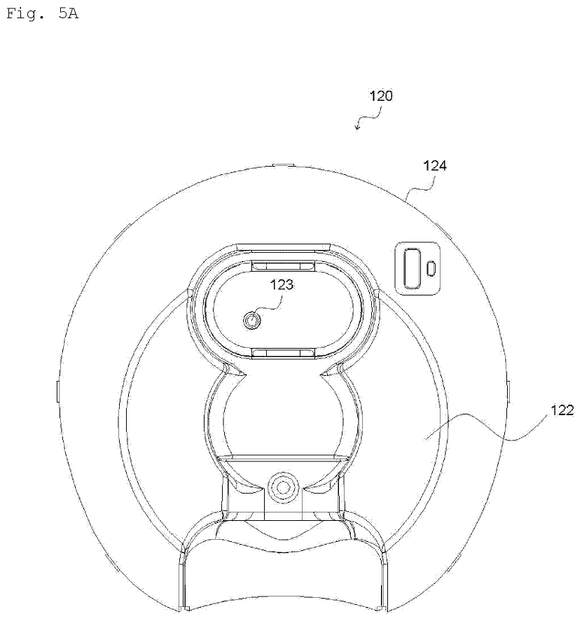

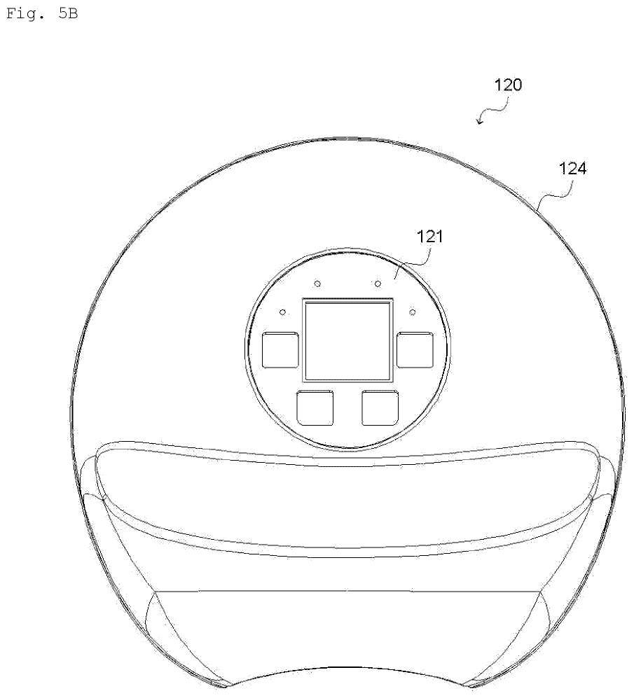

Through such the shape, the close contact unit 100 can come into close contact with the user's breast to allow an expression pressure for expressing the breast milk to be produced in an interior of the insertion groove 101, and as shown in FIG. 3, the breast milk introduced into the insertion groove 101 through the expression pressure flows to the internal passage 102 and is then discharged downward.

Further, the connection unit 110 is connected to the close contact unit 100 and has a relation with the production of the expression pressure in the close contact unit 100 and the discharge of the breast milk expressed. Together with the close contact unit 100, the connection unit 110 is necessarily provided for all of breast pumps for expressing breast milk.

In this case, close contact units may have similar shapes to one another in all of breast pumps, but the connection units may have various shapes different from one another.

In detail, as shown in FIG. 1A, 4A and 4B, the connection unit 110 includes a fastening groove 111 formed on one side thereof in such a manner as to insert the fastening portion 103 thereinto, an insertion groove 112 formed on the other side of an upper portion thereof with respect to the middle portion where the fastening groove 111 is formed in such a manner as to be connected to the fastening groove 111, a discharging passage 113 formed vertically downward therefrom in such a manner as to be connected to the fastening groove 111, and an outlet 114 connected to the discharging passage 113 in such a manner as to be open downward therefrom.

In detail, the connection unit 110 has a three-stage structure divided into upper, middle, and lower portions, and accordingly, as shown in FIG. 3, the expression unit 120 is connected to the upper portion of the connection unit 110 to allow the expression pressure to be produced in the insertion groove 101 and the internal passage 102. Further, a storage container 130 is connected to the lower portion of the connection unit 110, and the middle portion of the connection unit 110, which is connected to the upper and lower portions thereof, is connected to the close contact unit 100 to allow the expression pressure to be produced in the insertion groove 101 and the internal passage 102 and also to allow the expressed breast milk to flow into the storage container 130.

Further, the storage container 130 is a typical container that is connected to the lower portion of the connection unit 110 to store the breast milk flowing through the outlet 114 and is desirably made of a transparent plastic material so that an amount of breast milk stored therein can be easily checked at the outside.

In this case, the production of the expression pressure in the insertion groove 101 means that a pressure difference between an interior of the expression unit 120 and an interior of the insertion groove 101 is made to allow the breast milk to be expressed from the user's breast and thus to allow the expressed breast milk to flow toward the fastening portion 103 and the connection unit 110, and further, the production of the expression pressure in the internal passage 102 means that a pressure difference between the interior of the expression unit 120 and the interior of the internal passage 102 is made to allow the expressed breast milk to flow toward the connection unit 110.

Moreover, as shown in FIG. 1A, the breast pump according to the present invention includes a backflow prevention part 140 made of an elastic material and having the shape of a container with a ‘ [symbol] ’-shaped flange protruding outward therefrom, and as the ‘ [symbol] ’-shaped flange of the backflow prevention part 140 is fitted to an entrance of the insertion groove 112, the backflow prevention part 140 is insertedly fastened to the insertion groove 112 to prevent the backflow of the breast milk due to the expression pressure.

Furthermore, as shown in FIG. 1A, the breast pump according to the present invention includes a silicone head 150 with a cylindrical upper portion open on top and bottom thereof in such a manner as to be fastened to the end periphery of the discharging passage 113 and with a lower portion protruding downward from the open bottom of the upper portion thereof in such a manner as to be inclined downward from both side surfaces thereof and to have a straight line type open end portion. The silicone head 150 prevents the breast milk from flowing back from the storage container 130 toward the discharging passage 113.

Furthermore, the expression unit 120 is connected to the connection unit 110 to repeatedly produce the expression pressure patterned for the expression of the breast milk or for the breast massage, and as shown in FIG. 1C, 5A and 5B, the expression unit 120 includes a casing 124 coupled to the close contact unit 100 and having an accommodation groove 122 formed on one side thereof in such a manner as to be open on bottom thereof to accommodate the connection unit 110 therein and a through hole 123 protruding from an interior of the accommodation groove 122 to allow the inside thereof to communicate with the outside thereof, an expressing pump located inside the casing 124 to repeatedly produce the expression pressure, and a power supply part located inside the casing 124 to supply power.

Further, the expression unit 120 includes a production part having an input part 121 located on one side of the casing 124 to produce an input signal for power supply through the user's input and an input signal for the production of the expression pressure and the pressure adjustment, a controller for controlling the expressing pump and the power supply part, based on the input signals, to produce the expression pressures having various strengths and patterns adequate for the expression of the breast milk or the breast massage, and a storage part for storing only the input signals for the production of the expression pressure and the pressure adjustment among the produced input signals in order.

So as to express the breast milk, accordingly, the fastening portion 103 of the close contact unit 100 is inserted into the fastening groove 111 of the connection unit 110 to allow the close contact unit 100 and the connection unit 110 to be coupled to each other, and after the storage container 130 is coupled to the lower portion of the connection unit 110, next, the connection unit 110 is accommodated into the accommodation groove 122 of the casing 124 to allow the close contact unit 100, the connection unit 110, the expression unit 120, and the storage container 130 to be coupled integrally to one another. The power is applied to the breast pump through the power supply part, and expressing the breast milk can be performed by means of the input part 121.

If it is desired to have the breast massage, further, the fastening portion 103 of the close contact unit 100 is inserted into the fastening groove 111 of the connection unit 110 to allow the close contact unit 100 and the connection unit 110 to be coupled to each other, and next, the connection unit 110 is accommodated into the accommodation groove 122 of the casing 124 to allow the close contact unit 100, the connection unit 110, and the expression unit 120 to be coupled integrally to one another. The power is applied to the breast pump through the power supply part, and the breast massage can be performed by means of the input parts 121. Unlike the expression, the breast massage does not necessarily require the coupling between the connection unit 110 and the storage container 130.

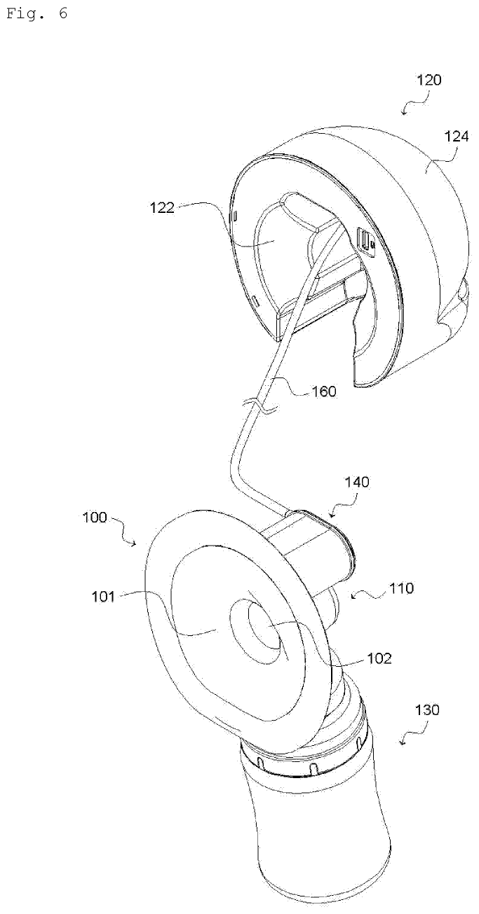

Further, as shown in FIG. 6, the breast pump according to the present invention includes an expression hose 160 whose one side end portion is connected to the through hole 123 and the other side end portion is connected to the insertion groove 112 of the connection unit 110. Accordingly, the smart breast pump according to the present invention can be used by the user who desires to express the breast milk or to have breast massage in an embodiment of the present invention wherein the close contact unit 100, the connection unit 110, the storage container 130, and the expression unit 120 are coupled integrally to one another. Under such a configuration with the expression hose 160, further, the smart breast pump according to the present invention can be used by the user who desires to express the breast milk or to have breast massage in another embodiment of the present invention wherein after the close contact unit 100, the connection unit 110, and the storage container 130 are coupled integrally to one another, the connection unit 110 and the expression unit 120 are connected to each other by means of the expression hose 160.

On the other hand, the input signal produced through the production part is divided into an input signal for driving the power supply part or stopping driving the power supply part and an input signal for controlling the expressing pump after the power supply part has been driven, and the input signal for controlling the expressing pump, that is, for producing the expression pressure is stored in the storage part in order of input, together with time differences of the input, which can be utilized for generating one massage course.

In more detail, the smart breast pump according to the present invention can select and input any one of an expression mode for expressing the breast milk and a massage mode for breast massage through the input part 121, and if the massage mode is selected in a state where a massage course is not generated, various kinds of input signals generated from the input part 121 are stored in order as time passes until a given set time elapses, thereby generating a new massage course.

Accordingly, the smart breast pump according to the present invention can generate the massage course optimized to the user through the input part 121.

Further, if the massage mode is selected in a state where a massage course is stored, the stored massage course is reproduced, and from the time point where a new input signal is generated while the stored massage course is being reproduced, furthermore, other input signals inclusive of the new input signal are stored in order as time passes to generate a new massage course.

Under the above-mentioned configuration, an embodiment of the present invention is as follows.

After a 30-minute massage mode is first selected by the user, any one of a plurality of buttons constituting the input part 121 is selected to repeatedly produce an expression pressure with a given strength (hereinafter referred to as ‘first strength’). After three minutes have elapsed, next, another button is selected to repeatedly produce an expression pressure with another given strength (hereinafter referred to as ‘second strength’), and after five minutes have elapsed, further, another button is selected to repeatedly produce an expression pressure with another given strength (hereinafter referred to as ‘third strength’).

In this case, one massage course (hereinafter referred to as ‘first course’) wherein the expression pressure with the first strength is repeatedly produced for three minutes, the expression pressure with the second strength is repeatedly produced for five minutes, and the expression pressure with the third strength is repeatedly produced for the remaining 22 minutes is stored to the storage part, and the stored massage course can be exactly reproduced through the controller if the input signal for power supply is generated again.

At the moment when five minutes elapse after the first course has started, further, any one of the plurality of buttons constituting the input part 121 is selected to repeatedly produce an expression pressure with a given strength (hereinafter referred to as ‘fourth strength’), and at the moment when five minutes elapse again, next, another button is selected to repeatedly produce an expression pressure with another given strength (hereinafter referred to as ‘fifth strength’). In this case, a new massage course wherein the expression pressure with the first strength is repeatedly produced for three minutes, the expression pressure with the second strength is repeatedly produced for two minutes, the expression pressure with the fourth strength is repeatedly produced for the five minutes, and the expression pressure with the fifth strength is repeatedly produced for the remaining 20 minutes is stored to the storage part, instead of the existing massage course.

If a new input signal is generated from the user while the first course is being reproduced, in detail, a new second course inclusive of a portion of the first course is generated and stored in the storage part.

Further, (the massage course stored in the storage part can be removed (hereinafter referred to as ‘first reset’) through a reset button of the input part 121, and the massage mode can be selected in the state where the stored massage course has been removed. In this case, the expression pressure with the lowest strength among the strengths the smart breast pump has is repeatedly produced for 30 minutes through the control of the controller.

If one or more buttons of the input part 121 are selected in order by the user, in this case, the expression pressures with the strengths corresponding to the buttons are repeatedly produced in order, and the expression pressure corresponding to the last button selected is stored in the storage part. If the input signal for power supply is generated again, after that, the expression pressure with the same strength as stored is exactly reproduced.

Furthermore, the massage course stored in the storage part can be removed again (hereinafter referred to as ‘second reset’) through the reset button of the input part 121, and the massage mode can be selected in the state where the stored massage course has been removed. In this case, one or more buttons of the input part 121 are selected in order to generate a new massage course.

If the first reset is generated, in detail, any one of the plurality of buttons constituting the input part 121 is selected to repeatedly produce the expression pressure with the first strength, another button after three minutes have elapsed is selected to repeatedly produce the expression pressure with the second strength, and another button after five minutes have elapsed is selected to repeatedly produce the expression pressure with the third strength. However, in this case, only the expression pressure with the third strength, which is lastly selected, is stored in the storage part. If the second reset is generated, one massage course wherein the expression pressure with the first strength is repeatedly produced for three minutes, the expression pressure with the second strength is repeatedly produced for five minutes, and the expression pressure with the third strength is repeatedly produced for the remaining 22 minutes is generated and stored in the storage part.

According to the present invention, on the other hand, time for one massage course is limited to 30 minutes, but it may be freely changed to 10, 15, and 20 minutes through the user's selection on the input part 121, without being limited thereto.

According to the present invention, further, if the input signal for stopping driving the power supply part is generated while a new massage course is being generated through the input part 121, a massage course inclusive of one or more input signals previously inputted is just generated, and if the input signal for stopping driving the power supply part is generated to stop producing the expression pressure while the massage course stored in the storage part is being reproduced, the massage course stored in the storage part is corrected to a massage course wherein patterns inputted after the production of the expression pressure has stopped are deleted.

After the massage course has been corrected, further, if the input signal for driving the power supply part is first generated, the expression pressure produced at the time point when the production of the expression pressure has stopped is produced exactly until the massage course is finished.

Under the above-mentioned configuration, an embodiment of the present invention is as follows.

After a 30-minute massage mode is first selected by the user, any one of the plurality of buttons constituting the input part 121 is selected to repeatedly produce an expression pressure with a given strength (hereinafter referred to as ‘first strength’). After three minutes have elapsed, next, another button is selected to repeatedly produce an expression pressure with another given strength (hereinafter referred to as ‘second strength’), and after five minutes have elapsed, further, another button is selected to repeatedly produce an expression pressure with another given strength (hereinafter referred to as ‘third strength’). After three minutes have elapsed, next, another button is selected to stop driving the power supply part.

In this case, one massage course (hereinafter referred to as ‘first course’) wherein the expression pressure with the first strength is repeatedly produced for three minutes, the expression pressure with the second strength is repeatedly produced for five minutes, and the expression pressure with the third strength is repeatedly produced for the remaining 22 minutes is stored to the storage part, and the stored massage course can be exactly reproduced through the controller if the input signal for power supply is generated again.

At the moment when five minutes elapse after the first course has started, further, any one of the plurality of buttons constituting the input part 121 is selected to stop driving the power supply part, and in this case, the massage course stored in the storage part is corrected to a massage course wherein the expression pressure with the first strength is repeatedly produced for three minutes and the expression pressure with the second strength is repeatedly produced for the remaining 27 minutes. If the input signal for driving the power supply part is generated again, the expression pressure with the second strength can be produced for 25 minutes.

The smart breast pump according to the present invention can allow the user to directly generate and repeatedly reproduce the expression or massage course optimized to her, allow the whole or portion of the expression or massage course reproduced to be corrected simply, and allow the corrected course to be reproduced again. Accordingly, the smart breast pump according to the present invention has remarkable differences from the conventional breast pumps in the functions for performing the expression or massage course and in the methods for performing the functions.

As described above, the smart breast pump with a massage course generating function according to the present invention is configured to select any one of the expression mode for expressing breast milk and the massage mode for the breast massage, so that if the massage mode is selected in a state where a massage course is not generated, various kinds of input signals generated from the input part are stored in order as time passes until a given set time elapses, thereby generating and storing a new massage course, and if the massage mode is selected in a state where a massage course is stored, the stored massage course is reproduced exactly, thereby allowing the user to directly generate and repeatedly reproduce the expression or massage course optimized to her.

In addition, the smart breast pump with a massage course generating function according to the present invention is configured to store, from the time point where a new input signal is generated while the stored massage course is being reproduced, other input signals inclusive of the new input signal in order as time passes to generate another new massage course, so that the whole or portion of the course being reproduced can be corrected simply by the user and also the corrected course can be reproduced again.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

In the description, components having no relation with the explanation of the present invention are not shown in the drawings for the clarity of the description, and the widths, lengths, and thicknesses of the components shown in the drawing may be magnified or reduced.

In the description, further, the corresponding parts in the embodiments of the present invention are indicated by corresponding reference numerals.

What is claimed is:

1. A smart breast pump with a massage course generating function, comprising:
a close contact unit (100) attached to a user's breast;
an expression unit (120) for repeatedly producing expression pressures patterned by the user's input to control an internal pressure of the close contact unit (100); and
a connection unit (110) for connecting the close contact unit (100) to the expression unit (120) to have a relation with the production of the expression pressures in the close contact unit (100) and the discharge of breast milk expressed,
wherein through an input part (121) mounted on the expression unit (120), any one of an expression mode for expressing the breast milk and a massage mode for massaging the breast is selected, so that if the massage mode is selected in a state where a massage course is not generated, various kinds of input signals generated through the input part (121) are stored in order as time passes until a given set time elapses thus to generate and store a new massage course; if the massage mode is selected in a state where a massage course is stored, the stored massage course is reproduced; and from the time point where a new input signal is generated while the stored massage course is being reproduced, other input signals inclusive of the new input signal are stored in order as time passes to thus generate another new massage course.

2. The smart breast pump according to claim 1, wherein the close contact unit (100) has the shape of a cone with an insertion groove (101) formed on one side thereof and a fastening portion (103) protruding from the other side thereof to allow an end portion of an internal passage (102) connected to the insertion groove (101) to be open downward therefrom, the connection unit (110) comprises a fastening groove (111) formed on one side thereof in such a manner as to insert the fastening portion (103) thereinto, an insertion groove (112) formed on the other side of an upper portion thereof with respect to the middle portion where the fastening groove (111) is formed in such a manner as to be connected to the fastening groove (111), a discharging passage (113) formed vertically downward therefrom in such a manner as to be connected to the fastening groove (111), and an outlet (114) connected to the discharging passage (113) in such a manner as to be open downward therefrom, and the expression unit (120) is connected to the connection unit (110) to repeatedly produce the expression pressures patterned through the user's input and thus to adjust the internal pressure of the insertion groove (101).

3. The smart breast pump according to claim 2, wherein the expression unit (120) comprises:
a casing (124) coupled to the close contact unit (100) and having an accommodation groove (122) formed on one side thereof in such a manner as to be open on the bottom thereof to accommodate the connection unit (110) therein and a through hole (123) protruding from an interior of the accommodation groove (122) to allow the inside thereof to communicate with the outside thereof;
an expressing pump located inside the casing (124) to repeatedly produce the expression pressures;
a power supply part located inside the casing (124) to supply power;
a production part having the input part (121) located on one side of the casing (124) to produce an input signal for power supply through the user's input and an input signal for the production of expression pressure and the pressure adjustment adequate for the expression of the breast milk and the breast massage;
a controller for controlling the expressing pump and the power supply part, based on the input signals, to produce the expression pressures having various strengths and patterns adequate for the expression of the breast milk or the breast massage; and
a storage part for storing only the input signals for the production of expression pressure and the pressure adjustment among the input signals produced in order.

4. The smart breast pump according to claim 1, wherein if an input signal for stopping driving the power supply part is generated while a new massage course is being generated through the input part (121), a massage course inclusive of one or more input signals previously inputted is immediately generated and stored; if an input signal for stopping driving the smart breast pump is generated to stop producing the expression pressure while the massage course stored is being reproduced, the massage course stored is corrected to a massage course wherein patterns inputted after the production of the expression pressure has stopped are deleted; and after the massage course has been corrected, if an input signal for driving the smart breast pump is first generated, the expression pressure produced at the time point when the production of the expression pressure has stopped is produced exactly until the massage course is finished.
